(19) United States
(12) Patent Application Publication
(10) Pub. No.: US 2021/0287808 A1
(43) Pub. Date: Sep. 16, 2021

(54) CONDITION INFORMATION GENERATION DEVICE, CONDITION INFORMATION GENERATION METHOD AND COMPUTER-READABLE MEDIUM

(71) Applicant: NIHON KOHDEN CORPORATION, Tokyo (JP)

(72) Inventors: Takayuki SUGIYAMA, Tokorozawa-shi (JP); Akihiko YOSHIMURA, Tokorozawa-shi (JP)

(73) Assignee: NIHON KOHDEN CORPORATION, Tokyo (JP)

(21) Appl. No.: 17/188,590

(22) Filed: Mar. 1, 2021

(30) Foreign Application Priority Data
Mar. 12, 2020 (JP) 2020-043129

(51) Int. Cl. G16H 50/30; G16H 50/20; G16H 10/60

(52) U.S. Cl. CPC G16H 50/30; G16H 10/60; G16H 50/20

(57) ABSTRACT

A condition information generation device includes: an acquisition unit configured to acquire physiological information of a subject; and a generator configured to generate condition information of the subject based on the physiological information. The generator is configured to generate the condition information based on the physiological information acquired at a target time that is set arbitrarily, the target time being a time that is earlier than a time when the condition information generation device generates the condition information.

### CROSS-REFERENCE TO RELATED APPLICATIONS

This application is based on and claims priority under 35 USC 119 from Japanese Patent Application No. 2020-043129 filed on Mar. 12, 2020, the contents of which are incorporated herein by reference.

### TECHNICAL FIELD

The presently disclosed subject matter relates to a condition information generation device, a condition information generation method, and a non-transitory computer-readable medium.

### BACKGROUND

In related art, for example, a scoring method such as national early warning score (NEWS) or quick SOFA (qSOFA) is known as a method for discriminating a condition of a subject (see JP-T-2015-522822).

When the condition of the subject is discriminated by NEWS or the like, a score is calculated based on a plurality of evaluation items (parameters) at the time when the score is calculated. The plurality of parameters are acquired constantly or at predetermined intervals, and the score is calculated as necessary.

Here, it is assumed that a medical worker (for example, a ward nurse) determines that the condition of the subject may have deteriorated based on the acquired parameters or the like. In this case, the medical worker may intend to seek a determination from another medical worker who can discriminate the condition of the subject at a high level (for example, a certified nurse or doctor).

The other medical worker who has more advanced knowledge and skills discriminates the condition of the subject based on a score of NEWS or the like. The other medical worker calculates the score based on parameters at the time when the condition of the subject is discriminated, and discriminates the condition of the subject.

However, the score calculated by the other medical worker does not always accurately indicate the condition of the subject at the time when the medical worker determines that the condition of the subject may have deteriorated. In other words, there is a discrepancy between the time when the initial medical worker (for example, the ward nurse) calculates the score and the time when the other medical worker (certified nurse or doctor) calculates the score. In this respect, there is room for improvement.

An object of the presently disclosed subject matter is to provide a condition information generation device, a condition information generation method, and a non-transitory computer-readable medium that can generate information indicating a condition at a time earlier than a time when condition information of the subject is generated, such as the time when the medical worker determines that the condition of the subject may have deteriorated.

### SUMMARY

A condition information generation device according to a first aspect of the presently disclosed subject matter includes: an acquisition unit configured to acquire physiological information of a subject; and a generator configured to generate condition information of the subject based on the physiological information. The generator is configured to generate the condition information based on the physiological information acquired at a target time that is set arbitrarily, the target time being a time that is earlier than a time when the condition information generation device generates the condition information.

A condition information generation method according to a second aspect of the presently disclosed subject matter includes: acquiring physiological information of a subject, and generating condition information of the subject based on the physiological information. The generating comprises generating the condition information based on the physiological information acquired at a target time that is set arbitrarily, the target time being a time that is earlier than a time when the condition information is generated.

A non-transitory computer-readable medium according to a third aspect of the presently disclosed subject matter stores a computer program for causing a computer to execute the condition information generation method according to the second aspect of the presently disclosed subject matter.

### BRIEF DESCRIPTION OF DRAWINGS

Exemplary embodiment(s) of the present invention will be described in detail based on the following figures, wherein:

FIG. 1 is a functional block diagram illustrating a condition information generation device according to an embodiment of the presently disclosed subject matter;

FIG. 2 illustrates a reference value of qSOFA, which is one discrimination method;

FIG. 3 illustrates a reference value of NEWS, which is another one discrimination method;

FIG. 4 illustrates a standardization table of each discrimination method;

FIG. 5 illustrates a standardization table configured to standardize each type of physiological information;

FIG. 6 is a flowchart illustrating a method of generating condition information and condition level information;

FIG. 7A illustrates an acquisition status of the physiological information;

FIG. 7B illustrates an acquisition status of the physiological information;

FIG. 8 is a flowchart illustrating a method of specifying the physiological information used in a first discrimination method;

FIG. 9 is a flowchart illustrating the method of specifying the physiological information used in the first discrimination method;

FIG. 10 is a flowchart illustrating the method of specifying the physiological information used in the first discrimination method;

FIG. 11 is a flowchart illustrating the method of generating the condition information and the condition level information;

FIG. 12A illustrates the acquisition status of the physiological information; and

FIG. 12B illustrates the acquisition status of the physiological information.

### DESCRIPTION OF EMBODIMENTS

Hereinafter, an example of an embodiment of the presently disclosed subject matter will be described with reference to the drawings.

### First Embodiment

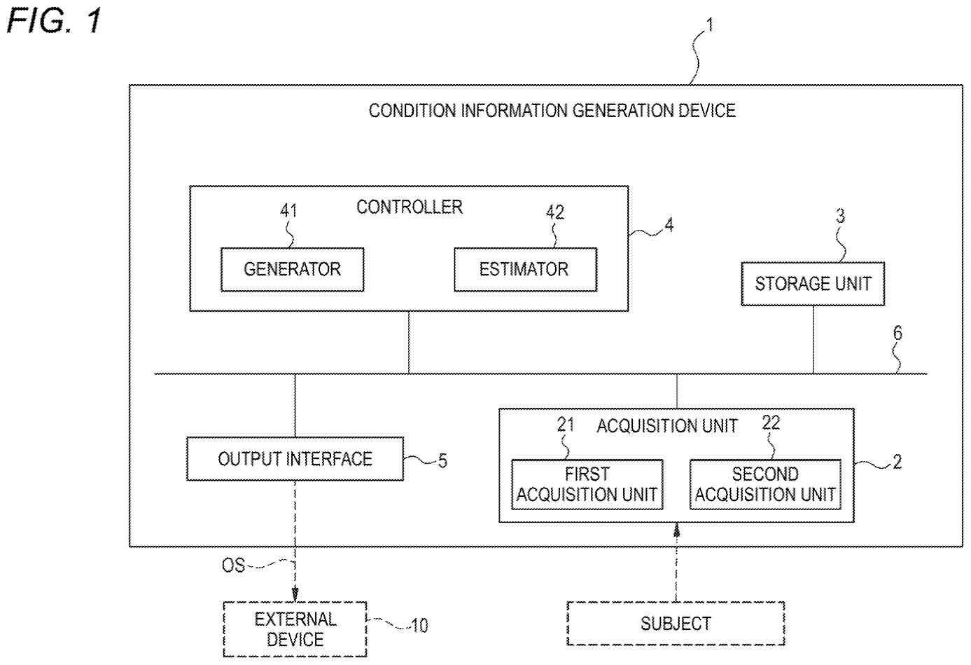

FIG. 1 illustrates a functional block diagram of a condition information generation device 1 according to the embodiment. As illustrated in FIG. 1, the condition information generation device 1 can include an acquisition unit 2, a storage unit 3, a controller 4, and an output interface (an example of an output unit) 5. Such components are communicably connected to each other via a bus 6.

The acquisition unit 2 is configured to acquire physiological information of a subject. The acquisition unit 2 can include a first acquisition unit 21 and a second acquisition unit 22. The first acquisition unit 21 acquires a continuous measurement parameter (an example of the physiological information) that is temporally continuously measured in a predetermined time range. The continuous measurement parameter refers to a parameter obtained by continuously measuring data from a sensor attached to the subject. The continuous measurement parameter is, for example, transcutaneous arterial oxygen saturation (SpO2), heart rate, or respiratory rate. The predetermined time range may be set as appropriate by a medical worker. The medical worker refers to, for example, a ward nurse, a certified nurse, or a doctor. Here, a case where a respiratory rate per minute is acquired will be described as an example. Examples of methods of acquiring the respiration rate include a measurement method based on end tidal CO2 (EtCO2), a measurement method based on breath sound, respiratory pressure or respiratory flow rate, a measurement method based on thoracic impedance, and an impedance type measurement method using an electrocardiogram.

For example, when the respiration rate is measured by the impedance type method using the electrocardiogram, a plurality of electrodes and indifferent electrodes are attached to the subject. Each electrode is in contact with a measurement site of the subject and functions as a sensor configured to synthesize a potential change of the measurement site. The electrode is configured to synthesize a potential difference of the measurement site, and the indifferent electrode is configured to remove external noise induced in phase with the electrode.

In the present example, an electrocardiogram signal is generated based on an electric signal indicating the potential change (potential) output from the electrode and an electric signal indicating the potential change (potential) output from the indifferent electrode. Then the generated electrocardiogram signal is subjected to analog-to-digital conversion (AD conversion) so as to generate electrocardiogram waveform data. The electrocardiogram waveform data refers to data indicating a plurality of electrocardiogram waveforms continuously generated on a time axis (waveforms generated by one beat). The generated electrocardiogram waveform data is transmitted to the first acquisition unit 21. In this way, the first acquisition unit 21 acquires the electrocardiogram waveform data of the subject and the heart rate or respiratory rate based on the electrocardiogram waveform data. The first acquisition unit 21 may also be configured to receive the electrocardiogram signal from the electrode and acquire the electrocardiogram waveform data by performing the analog-to-digital conversion (AD conversion) on the received electrocardiogram signal. The first acquisition unit 21 transmits the acquired electrocardiogram waveform data and the like to the storage unit 3.

The second acquisition unit 22 acquires a discontinuous measurement parameter (another example of the physiological information) measured at arbitrary timings. The discontinuous measurement parameter refers to a parameter that is measured in a spot-wise manner from the sensor attached to the subject, such as non-invasive blood pressure measurement or round visit body temperature measurement. The discontinuous measurement parameter is, for example, oxygen supplementation, body temperature, systolic blood pressure, or consciousness level. Here, a case where blood pressure is acquired in one specific minute will be described as an example. A method of acquiring the blood pressure is, for example, a non-invasive blood pressure measurement method using a cuff.

For example, when the systolic blood pressure is measured by the non-invasive blood pressure measurement method using the cuff, the cuff is wrapped around an upper arm of the subject, air pressure in the cuff is increased so as to block a blood flow of the subject, and thus an artery is compressed to measure the blood pressure. Then the measured blood pressure is subjected to the analog-to-digital conversion (AD conversion) to generate blood pressure data. The blood pressure data is transmitted to the second acquisition unit 22. The second acquisition unit 22 may also be configured to receive the blood pressure signal from a blood pressure sensor such as the cuff and acquire the blood pressure data by performing the analog-to-digital conversion (AD conversion) on the received blood pressure signal. In this way, the second acquisition unit 22 acquires the blood pressure data of the subject. The second acquisition unit 22 transmits the acquired blood pressure data to the storage unit 3.

The storage unit 3 stores a condition discrimination method of discriminating a condition of the subject, a reference value or permissible range defined in a medical guideline or the like related to the condition discrimination method, a standardization table configured to standardizing each discrimination result, the physiological information of the subject, and the like. Examples of the condition discrimination method include, for example, qSOFA, NEWS, SOFA, acute physiology and chronic health evaluation 2 (APACHE 2), bedside shivering assessment scale (BSAS), and national institutes of health stroke scale (NIHSS). The condition discrimination method stored in the storage unit 3 may also include, for example, a unique condition discrimination method set based on NEWS or the like in addition to the such condition determination methods.

The controller 4 can include a generator 41 and an estimator 42. The controller 4 can include one or more memories and one or more processors as hardware configurations. The memory is constituted by, for example, a read-only memory (ROM) in which various programs and the like are stored, or a random access memory (RAM) including a plurality of work areas in which various programs executed by the processor and the like are stored. The processor is, for example, a central processing unit (CPU), and is configured to load a program specified from various programs incorporated in the ROM onto the RAM and execute various types of processing in cooperation with the RAM. For example, the controller 4 performs control such that processing of the generator 41 or the estimator 42 is implemented by executing the program by the processor of the controller 4 in cooperation with the RAM.

Here, the program can be stored and provided to a computer through using various types of non-transitory computer-readable media. The non-transitory computer-readable media include various types of tangible storage media. Examples of the non-transitory computer-readable media include magnetic recording media (for example, flexible disks, magnetic tapes, hard disk drives), magneto-optical recording media (for example, magneto-optical disks), CD-ROM (read only memory), CD-R, CD-R/W, and semiconductor memories (for example, mask ROMs, programmable ROMs (PROM), erasable PROMs (EPROM), flash ROMs). The program may also be provided to the computer by various types of transitory computer-readable media. The transitory computer-readable media are, for example, electric signals, optical signals, and electromagnetic waves. The transitory computer-readable media can provide the program to the computer via a wired communication path such as electric wires and optical fibers, or a wireless communication path.

The generator 41 is configured to generate the condition information based on the physiological information of the subject acquired by the acquisition unit 2, the condition discrimination method and the reference value defined by the medical guideline and the like stored in the storage unit 3. The condition information refers to information on the condition of the subject. The information on the condition of the subject is, for example, a score calculated based on the condition discrimination method. The generator 41 can also generate the condition level information of the subject based on the condition information and the standardization table. The condition level information is, for example, a grade indicating a degree of the condition of the subject. The generator 41 transmits the generated condition information or condition level information to the output interface 5.

The estimator 42 is configured to estimate an estimated discontinuous measurement parameter (an example of the discontinuous measurement parameter), which is an estimated value of the discontinuous measurement parameter at a time when the discontinuous measurement parameter is not acquired, based on the discontinuous measurement parameter acquired by the second acquisition unit 22. The discontinuous measurement parameter used for estimation of the estimated discontinuous measurement parameter can be arbitrarily selected, and it is preferable to use one or more discontinuous measurement parameters acquired at a time relatively close to a time when the estimated discontinuous measurement parameter is to be estimated among times when the discontinuous measurement parameters are acquired.

The output interface 5 is configured to output an output signal OS corresponding to the condition information or the condition level information. For example, the output interface 5 can transmit the output signal OS to an external device 10 which is a device different from the condition information generation device 1. If necessary, the output interface 5 can include a circuit configured to convert output data into the output signal OS that can be processed by the external device 10.

The external device 10 is, for example, a display device including a display unit, such as a liquid crystal display or an organic EL display, or a mobile terminal. Upon receiving the output signal OS from the output interface 5, the external device 10 displays the condition information or the condition level information of the subject corresponding to the output signal OS on the display unit included in the external device 10.

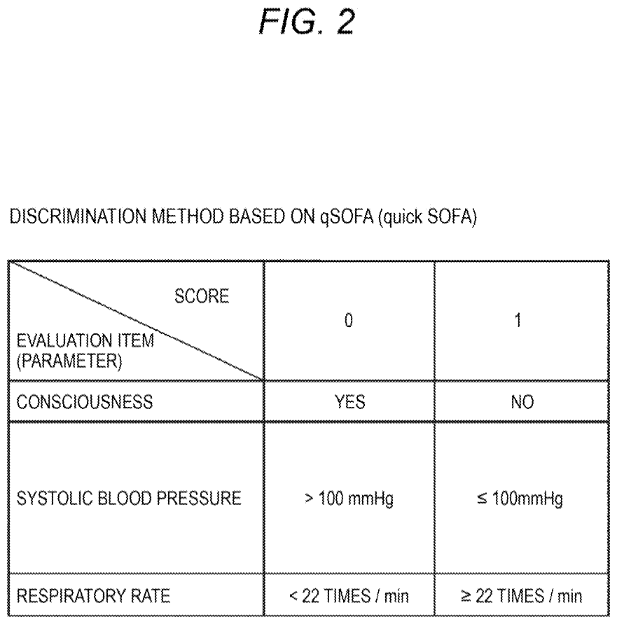

Next, the condition information and the condition level information of the subject generated by the generator 41 will be described in detail with reference to FIGS. 2 to 5. FIG. 2 illustrates a reference value of qSOFA, which is one condition discrimination method. qSOFA is a discrimination method used to discriminate septicemia. qSOFA is used especially when discriminating whether an infectious disease is present. For example, when the acquired respiratory rate per minute is 22 or more, a score related to the respiratory rate is 1. Otherwise, the score is 0. When the acquired systolic blood pressure is 100 mmHg or less, a score related to the systolic blood pressure is 1. Otherwise, the score is 0. Further, when the subject is unconscious, a score related to consciousness is 1. On the other hand, when the subject is conscious, the score related to the consciousness is 0. The calculated scores are summed. In the present specification, a total score thereof is referred to as a total score A (an example of the condition information).

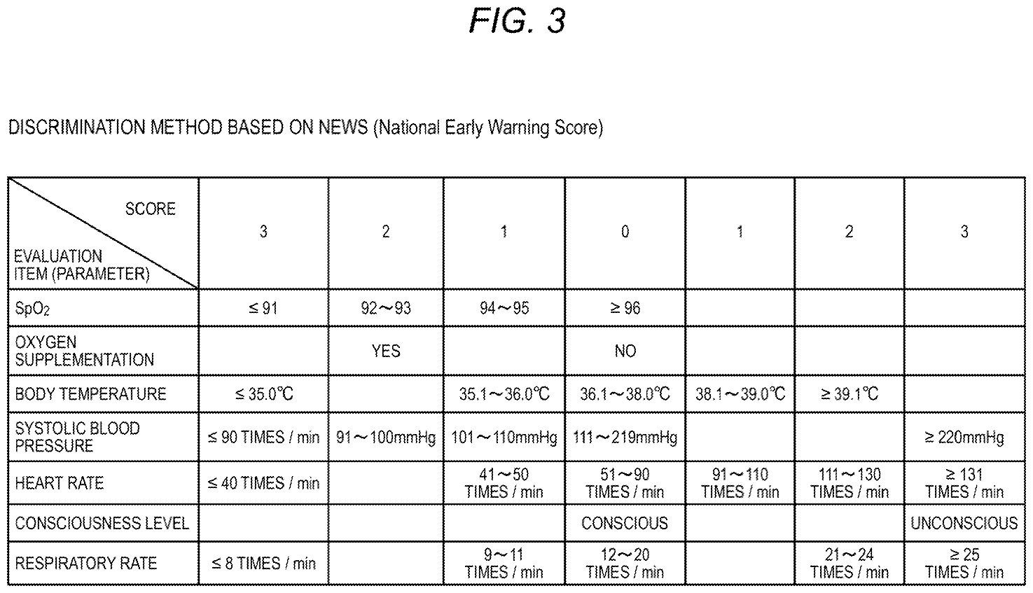

FIG. 3 illustrates the reference value of NEWS, which is another one condition discrimination method. NEWS is a discrimination method used to discriminate septicemia. When the acquired respiratory rate per minute is equal to or less than 8 or equal to or greater than 25, the score related to the respiratory rate is 3. When the respiratory rate is equal to or greater than 21 and equal to or less than 24, the score related to the respiratory rate is 2. When the respiratory rate is equal to or greater than 9 and equal to or less than 11, the score related to the respiratory rate is 1. When the respiratory rate is equal to or greater than 12 and equal to or less than 20, the score related to the respiratory rate is 0. Scores for remaining parameters are also calculated based on the reference value defined by the medical guideline or the like. The calculated scores are summed. In the present specification, a total score thereof is referred to as a total score B (another example of the condition information).

The scores described above are calculated and summed up by the generator 41. Although the total score A and the total score B are numerical values in the present embodiment, the total scores may also be represented by other expression forms such as percentages.

In this way, for example, medical meaning of the reference value and values of the total scores is different for each discrimination method even though the discrimination methods are all related to septicemia. Therefore, in the present embodiment, the condition level information is generated based on the standardization table configured to standardize the condition information, such as the condition information and the total score of each discrimination method.

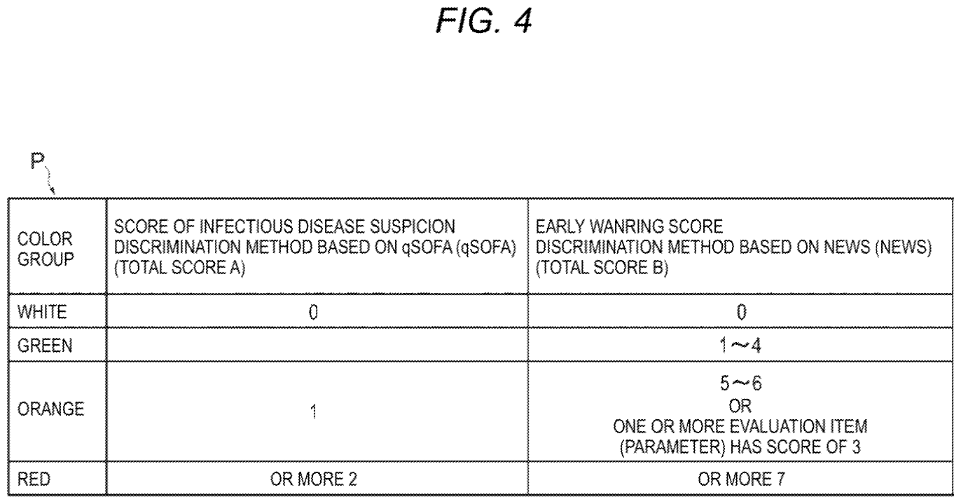

FIG. 4 illustrates a standardization table P of each discrimination method. The standardization table P is a table configured to display condition levels of the subject by classifying the condition levels into four color groups for each discrimination method. The four color groups in the present embodiment include a white group, a green group, an orange group, and a red group. The white group corresponds to a condition level indicating that the subject is in a normal state. The red group corresponds to a condition level indicating that the subject is in an abnormal state and is in a dangerous state. The orange group corresponds to a condition level indicating that the subject is not in the normal state though the subject is not as dangerous as the subject of the red group. The green group corresponds to a condition level indicating that the subject is not in the normal state though the subject is not as dangerous as the subject of the orange group. That is, the condition level of the subjects other than the white group becomes worse in the order of the green group, the orange group, and the red group. The classification of the color groups described here is merely an example. It is needless to say that the classification of the color groups is not limited to this example.

For example, when the total score A of the score (qSOFA) indicating infectious disease suspicion is 0, a color group thereof is classified into the white group. When the total score A is 1, the color group is classified into the orange group. When the total score A is 2 or more, the color group is classified into the red group.

For example, when the total score B of the early warning score (NEWS) is 0, a color group thereof is classified into the white group. When the total score B is equal to or greater than 1 and equal to or less than 4, the color group is classified into the green group. When the total score B is equal to or greater than 5 and equal to or less than 6, or at least one evaluation item of NEWS has a score of 3, the color group is classified into the orange group. When the total score B is 7 or more, the color group is classified into the red group.

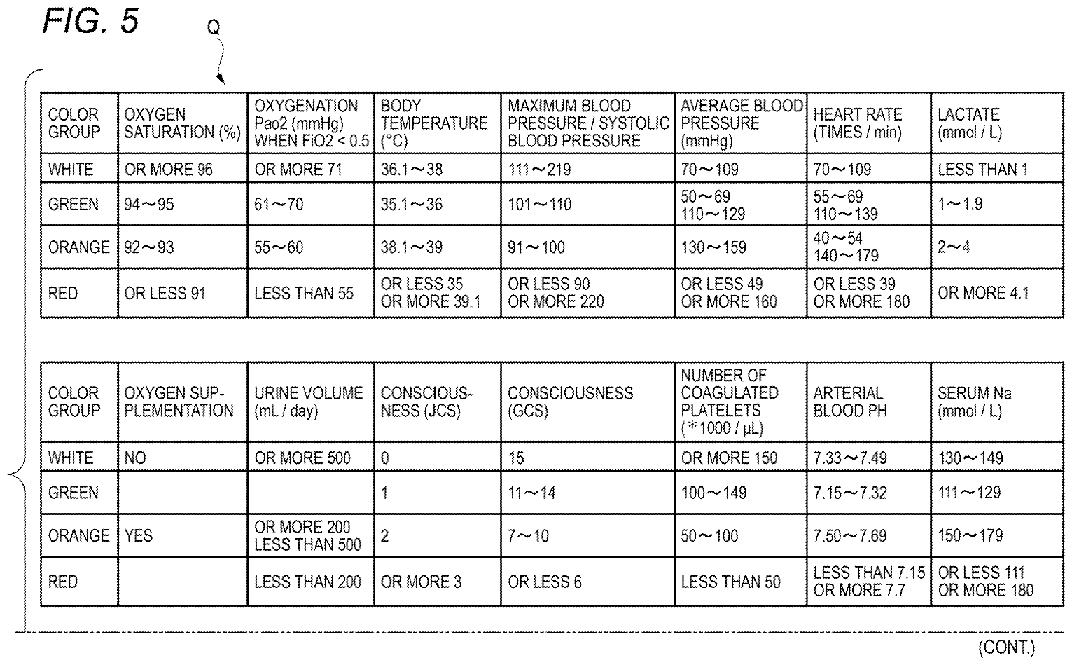

FIG. 5 illustrates a standardization table Q configured to standardize each type of physiological information. As illustrated in FIG. 5, for example, when the respiratory rate per minute is equal to or greater than 12 and equal to or less than 20, a color group thereof is classified into the white group. When the respiratory rate is equal to or greater than 9 and equal to or less than 11, the color group is classified into the green group. When the respiratory rate is equal to or greater than 21 and equal to or less than 24, the color group is classified into the orange group. When the respiratory rate is equal to or less than 8 or equal to or greater than 25, the color group is classified into the red group.

As illustrated in FIG. 5, a color group concerning the oxygen supplementation is classified into the orange group when the oxygen supplementation is required, and the color group is classified into the white group when the oxygen supplementation is not required. A color group concerning urine volume is classified into the white group when urine volume per day is 500 mL or more. The color group is classified into the orange group when the urine volume per day is equal to or greater than 200 mL and less than 500 mL. The color group is classified into the red group when the urine volume per day is less than 200 mL. Other types of physiological information are also classified into four stages, and one color group is associated with each stage.

The classification described above is performed by the generator 41. That is, the generator 41 classifies the condition level of the subject based on each total score or each type of physiological information, and the standardization table P or the standardization table Q stored in the storage unit 3. For example, when qSOFA or NEWS is used to discriminate whether the subject has septicemia, the generator 41 discriminates which color group of the four color groups the condition level of the subject is classified into based on the total score A or the total score B and the standardization table P stored in the storage unit 3, and generates the condition level information of the subject in accordance with a discrimination result thereof.

### First Example

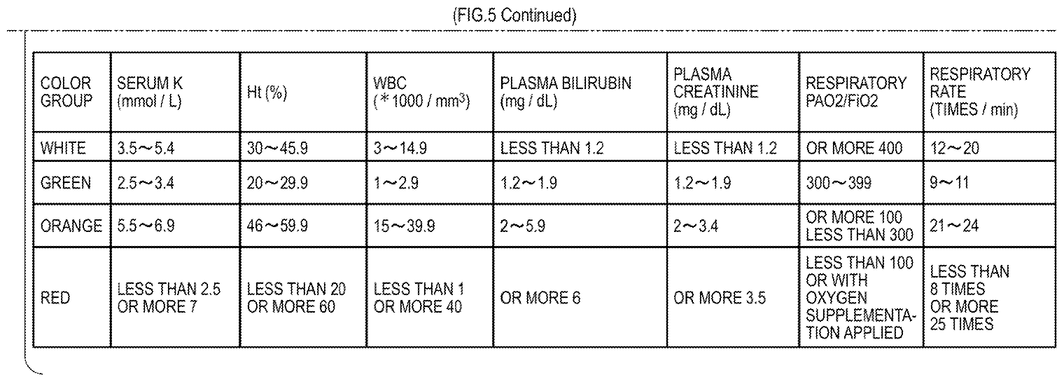

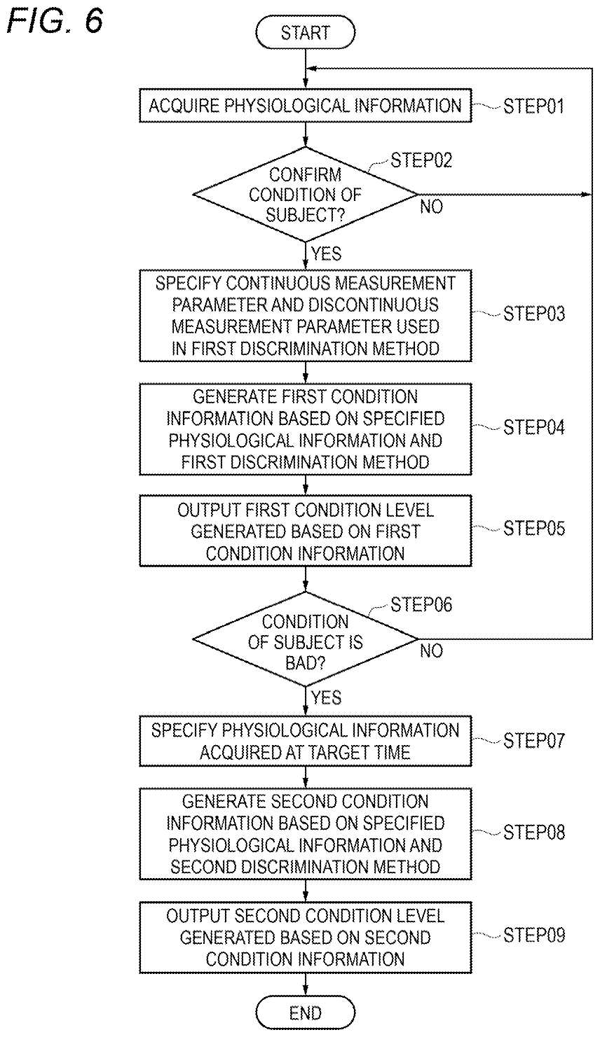

Next, a method of generating the condition information and the condition level information will be described in detail with reference to FIGS. 6 to 8. In the present embodiment, an example, in which the condition of the subject whose name is Kodentaro is discriminated by two different condition discrimination methods (qSOFA and NEWS) at different times, is used for description. In the present embodiment, after a ward nurse discriminates the condition of Kodentaro by qSOFA at 9:40 on Jul. 10, 2017, a certified nurse or doctor discriminates the condition of Kodentaro by NEWS at 10:00 on Jul. 10, 2017. This is because qSOFA requires less parameter and is simpler as compared with NEWS, while NEWS has higher accuracy than qSOFA and requires more parameters, and certified nurses and doctors are generally considered to be capable of performing condition discrimination at a higher degree as compared with ward nurses.

In the present embodiment, qSOFA, which is the condition discrimination method used first, serves as a first discrimination method, and NEWS, which is the condition discrimination method used after qSOFA, serves as a second discrimination method. Further, in the present specification, condition information generated based on the first discrimination method is referred to as first condition information and condition information generated based on the second discrimination method is referred to as second condition information.

In the present embodiment, a time that is set arbitrarily by a medical worker or the like is referred to as a target time, which is a time that is earlier than a time when the condition information generation device 1 generates the condition information. When a plurality of pieces of condition information are generated at a plurality of different times, for example, the first condition information is generated first, and then the second condition information is generated. In this case, a time that is set arbitrarily by the medical worker or the like, which is a time that is earlier than a time when the second condition information is generated, is referred to as the target time. The target time is, for example, a reference time for determining the condition of the subject (hereinafter referred to as a first target time) such as a time when the ward nurse determines that the condition of the subject may have deteriorated, or a time closest to the first target time among times when the discontinuous measurement parameter is acquired (hereinafter referred to as a second target time).

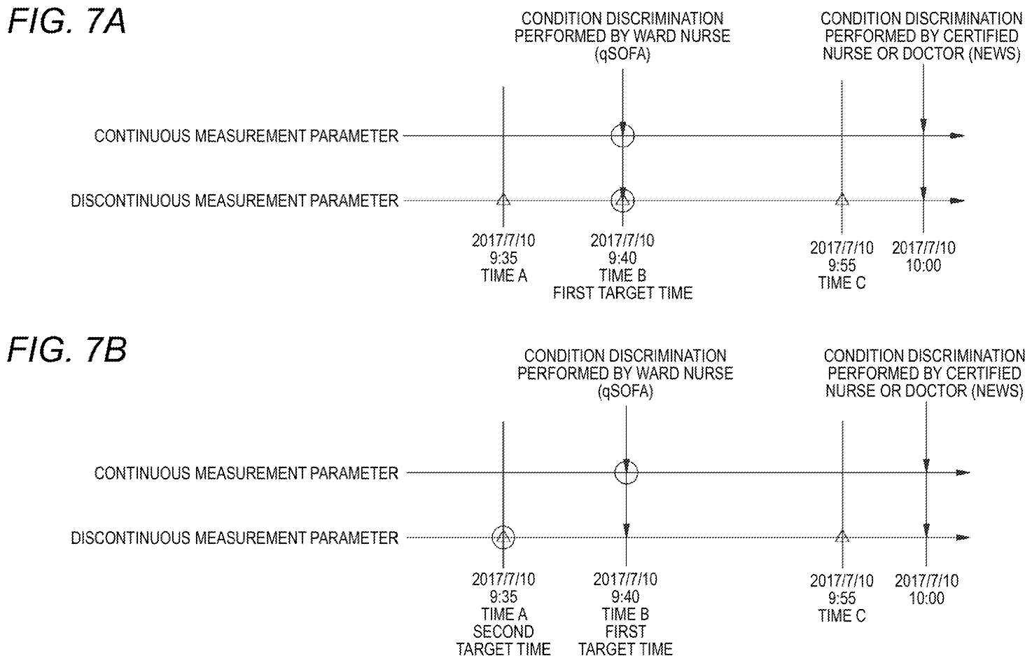

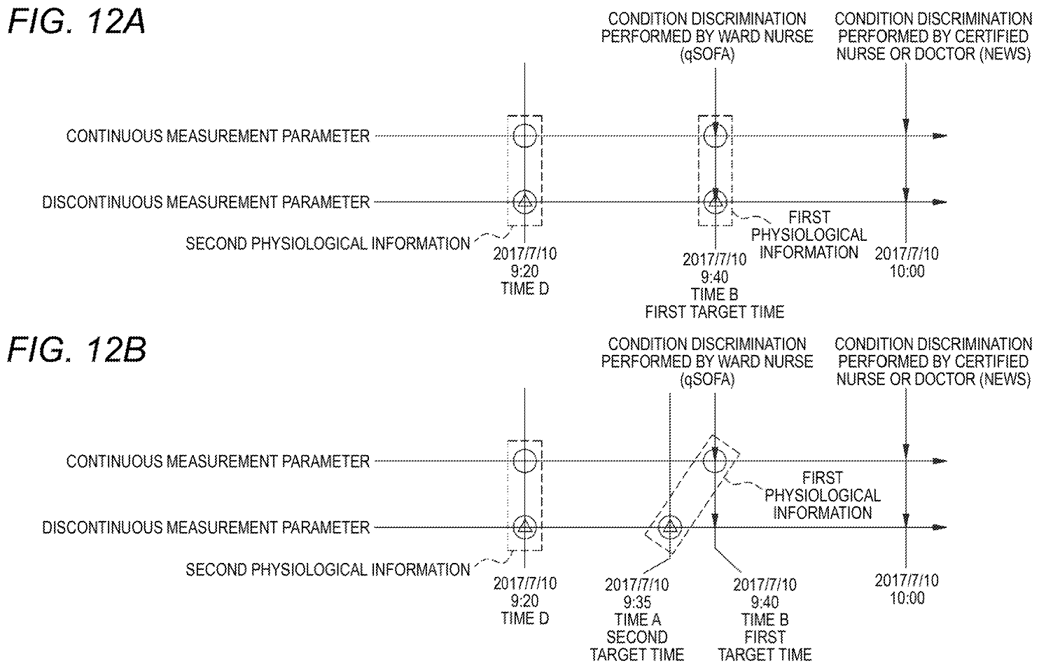

As illustrated in FIG. 6, first, the acquisition unit 2 of the condition information generation device 1 acquires the physiological information of the subject (Kodentaro) (STEP 01). At this time, the first acquisition unit 21 acquires the continuous measurement parameter, and the second acquisition unit 22 acquires the discontinuous measurement parameter. Here, when an acquisition status of the physiological information is as illustrated in FIG. 7A, the discontinuous measurement parameter is acquired only at 9:35 on Jul. 10, 2017 (time A), 9:40 on Jul. 10, 2017 (time B), and 9:55 on Jul. 10, 2017 (time C). Meanwhile, the continuous measurement parameter is continuously acquired not only at the times A to C but also at other times. When the acquisition status of the physiological information is as illustrated in FIG. 7B, the discontinuous measurement parameter is acquired only at the time A and the time C. Meanwhile, the continuous measurement parameter is continuously acquired not only at the time A and the time C but also at other times. In FIGS. 7A and 7B, the times when the discontinuous measurement parameter is acquired are indicated by triangles (the same also applies to FIGS. 12A and 12B).

In STEP 2, the medical worker determines whether it is necessary to confirm the condition of the subject. For example, when the ward nurse pays a round visit (9:40 on Jul. 10, 2017 in the present embodiment) to Kodentaro and determines that the condition of Kodentaro may have deteriorated and it is necessary to confirm the condition of Kodentaro, the ward nurse performs a predetermined input operation on a device such as a bedside monitor linked to Kodentaro. When the input operation is performed, condition confirmation information indicating that it is necessary to confirm the condition of the subject and information on the time (first target time) when the ward nurse determines that the condition of the subject may have deteriorated and performs the input operation are generated, and such pieces of information are transmitted to the condition information generation device 1 by wire or wirelessly. The condition information generation device 1 may be, for example, a device such as a bedside monitor which includes an operation unit configured to receive the input operation of the medical worker and generate an instruction signal corresponding to the input operation. In this case, the ward nurse performs the predetermined input operation on the operation unit provided in the condition information generation device 1. When the input operation is performed, the condition information generation device 1 generates the condition confirmation information and the information on the time (first target time) when the ward nurse determines that the condition of the subject may have deteriorated and performs the input operation. When the input operation is performed (YES in STEP 02), the controller 4 specifies the continuous measurement parameter and the discontinuous measurement parameter used in the first discrimination method based on the received condition confirmation information (STEP 03). On the other hand, when the controller 4 determines that it is not necessary to confirm the condition of Kodentaro (NO in STEP 02), the process returns to STEP 01.

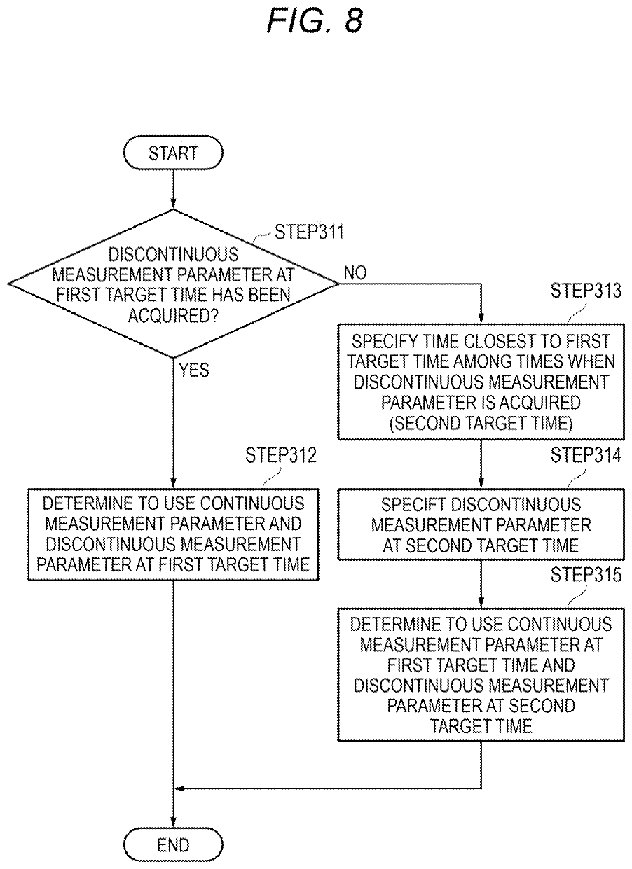

Here, STEP 03 will be described in detail with reference to FIG. 8. FIG. 8 is a flowchart illustrating a method of specifying the continuous measurement parameter and the discontinuous measurement parameter used in the first discrimination method. Since the continuous measurement parameter is temporally continuously measured, the continuous measurement parameter is also acquired at the first target time, while the discontinuous measurement parameter is not always acquired at the first target time since the discontinuous measurement parameter is measured at random timing. Therefore, in STEP 311, the controller 4 determines whether the discontinuous measurement parameter at the first target time is acquired. When the controller 4 determines that the discontinuous measurement parameter at the first target time is acquired, the process proceeds to STEP 312. On the other hand, if the controller 4 determines that the discontinuous measurement parameter at the first target time is not acquired, the process proceeds to STEP 313.

For example, when the acquisition status of the physiological information is as illustrated in FIG. 7A, the discontinuous measurement parameter is acquired at the time B which is the first target time. Therefore, the controller 4 determines that the discontinuous measurement parameter at the first target time is acquired (YES in STEP 311). When it is determined that the discontinuous measurement parameter at the first target time is acquired, the controller 4 determines to use the continuous measurement parameter and the discontinuous measurement parameter at the first target time in the first discrimination method (STEP 312).

On the other hand, for example, when the acquisition status of the physiological information is as illustrated in FIG. 7B, the discontinuous measurement parameter is not acquired at the time B which is the first target time. Therefore, the controller 4 determines that the discontinuous measurement parameter at the first target time is not acquired (NO in STEP 311). When it is determined that the discontinuous measurement parameter at the first target time is not acquired, the controller 4 specifies the second target time, which is the time closest to the first target time among the times when the discontinuous measurement parameter is acquired (STEP 313). Among the time A and the time C, the time closest to the first target time (time B) is the time A. Therefore, the controller 4 specifies the time A as the second target time.

When the second target time is specified, the controller 4 specifies the discontinuous measurement parameter at the second target time (STEP 314). When the discontinuous measurement parameter at the second target time is specified, the controller 4 determines to use the continuous measurement parameter at the first target time and the discontinuous measurement parameter at the second target time in the first discrimination method (STEP 315).

Referring back to FIG. 6, each STEP of STEP 04 and subsequent steps will be described. When the continuous measurement parameter and the discontinuous measurement parameter used in the first discrimination method are specified, the generator 41 generates the first condition information (total score A) based on the specified continuous measurement parameter and discontinuous measurement parameter, and the first discrimination method (STEP 04).

The generator 41 generates the first condition information, and then generates first condition level information (an example of the condition level information) of Kodentaro based on the first condition information and the standardization table P (see FIG. 4). The generator 41 generates the first condition level information, and then outputs the output signal OS corresponding to the first condition level information to the output interface 5 (STEP 05). The output interface 5 outputs the output signal OS to the external device 10. The external device 10 displays the condition level information of Kodentaro corresponding to the output signal OS on the display unit included in the external device 10. When the condition level information of Kodentaro is displayed on the display unit included in the external device 10, the condition level of Kodentaro is visually notified to the ward nurse. For example, when a color indicating that the condition of Kodentaro is bad (for example, red) is displayed on the display unit included in the external device 10, the ward nurse recognizes that the condition of Kodentaro is bad. A method of notifying the ward nurse is not limited to the visual method, and may also be an auditory method such as issuing a warning sound, for example. The display unit may also be built in the condition information generation device 1, and the condition level information or the like may be displayed on the display unit.

The ward nurse determines whether the condition of Kodentaro is bad (STEP 06). When the ward nurse determines that the condition of Kodentaro is not bad (NO in STEP 06), the process returns to STEP 01. On the other hand, if the ward nurse determines that the condition of Kodentaro is bad (YES in STEP 06), the ward nurse requests the certified nurse or doctor to further discriminate the condition of Kodentaro. Although the certified nurse or doctor discriminates the condition of the subject in response to the request, it is common that the ward nurse and the certified nurse or doctor are located at different places at the first target time. Therefore, a time when the certified nurse or doctor determines the condition of the subject by the second discrimination method is temporally later than the time when the ward nurse discriminates the condition of the subject by qSOFA (first discrimination method). That is, there is a time lag between the time (10:00 on Jul. 10, 2017) when the certified nurse or doctor discriminates the condition of the subject by NEWS (second discrimination method) and the time (9:40 on Jul. 10, 2017) when the ward nurse determines the condition of the subject by qSOFA (first discrimination method). Therefore, in related art, a time when physiological information used for qSOFA is acquired and a time when physiological information used for NEWS is acquired are different. Therefore, the total score B calculated by the certified nurse or the like does not always accurately indicate the condition of the subject at the time (first target time) when the ward nurse determines that the condition of the subject may have deteriorated. Therefore, in the present embodiment, the second discrimination method is performed based on the physiological information acquired at the same time as the time when the physiological information used in the first discrimination method is acquired. Such a mechanism will be described in detail in STEP 07 and subsequent steps.

When it is determined that the condition of Kodentaro is bad (YES in STEP 06), the ward nurse requests the certified nurse or doctor to perform further condition discrimination. The certified nurse or doctor goes to a place where Kodentaro is located at 10:00 on Jul. 10, 2017 to confirm the condition of Kodentaro. When the certified nurse or doctor determines that it is necessary to calculate the total score B (that is, when it is determined that it is necessary to discriminate the condition of Kodentaro by NEWS), the certified nurse or doctor performs a predetermined input operation on a device such as the bedside monitor linked to Kodentaro. When the input operation is performed, additional discrimination information indicating that the certified nurse or doctor further discriminates the condition of the Kodentaro, and information on a time when the input operation is performed are generated, and such pieces of information are transmitted to the condition information generation device 1 by wire or wirelessly. Upon receiving the additional discrimination information, the controller 4 specifies the physiological information acquired at the target time specified in STEP 03 (STEP 07). The specified physiological information not only includes the physiological information used in the first discrimination method, but also includes other physiological information (for example, heart rate or body temperature) used in the second discrimination method.

For example, when the acquisition status of the physiological information is as illustrated in FIG. 7A, the controller 4 specifies the continuous measurement parameter and the discontinuous measurement parameter acquired at the first target time. On the other hand, when the acquisition status of the physiological information is as illustrated in FIG. 7B, the controller 4 specifies the continuous measurement parameter acquired at the first target time and the discontinuous measurement parameter acquired at the second target time.

When the physiological information acquired at the target time is specified, the generator 41 generates the second condition information (total score B) based on the physiological information specified in STEP 07 and the second discrimination method (STEP 08).

The generator 41 generates the second condition information, and then generates second condition level information (another example of the condition level information) of Kodentaro based on the second condition information and the standardization table P. The generator 41 transmits the first condition level information and the second condition level information to the output interface 5 (STEP 09). The output interface 5 outputs the output signal OS corresponding to each condition level information. The output interface 5 transmits the output signal OS to the external device 10. The external device 10 displays the condition level information of Kodentaro corresponding to the output signal OS on the display unit included in the external device 10. The condition level information can be displayed on the display unit of the external device 10 in association with a color indicating the condition level of the subject, the subject, the time, and the like.

According to the above configuration, for example, when the condition of the subject is discriminated later than the target time such as the time when the medical worker determines that the condition of the subject may have deteriorated, the generator 41 can generate not only the total score A but also the total score B based on the physiological information of the subject at the target time. Therefore, the total score B calculated by the second discrimination method performed later than the target time also accurately indicates the condition of the subject at the target time. Therefore, the medical worker who discriminates the condition of the subject later than the target time can more accurately grasp the condition of the subject at the target time based on the total score B.

According to the above configuration, when the condition of the subject is discriminated based on the continuous measurement parameter and the discontinuous measurement parameter, the generator 41 can generate the condition information indicating the condition of the subject even if the target time set arbitrarily is a time when the discontinuous measurement parameter is not measured.

According to the above configuration, even when the first target time, which is the reference time among the target times set arbitrarily, is a time when the discontinuous measurement parameter is not measured, the generator 41 generates the condition information indicating the condition of the subject based on the continuous measurement parameter at the first target time and the discontinuous measurement parameter at the second target time which is the time closest to the first target time. Therefore, even if the first target time is a time when the discontinuous measurement parameter is not measured, the generator 41 can generate appropriate condition information.

According to the above configuration, the generator 41 generates the condition information based on the discrimination method configured to discriminate the condition of the subject. When the generator 41 generates the condition information based on the discrimination method defined by, for example, the medical guideline of qSOFA or NEWS, the generated condition information is reliable information.

According to the above configuration, even when the condition of the subject is discriminated through using the different discrimination methods at the different times, the generator 41 can generate the first condition information and the second condition information based on the physiological information acquired at the target time.

According to the above configuration, even when the condition of the subject is discriminated based on qSOFA, which can discriminate the condition of the subject relatively easily, and then the condition of the subject is discriminated based on NEWS, which has higher accuracy than qSOFA, the generator 41 can generate the first condition information and the second condition information based on the physiological information acquired at the target time.

### Second Example

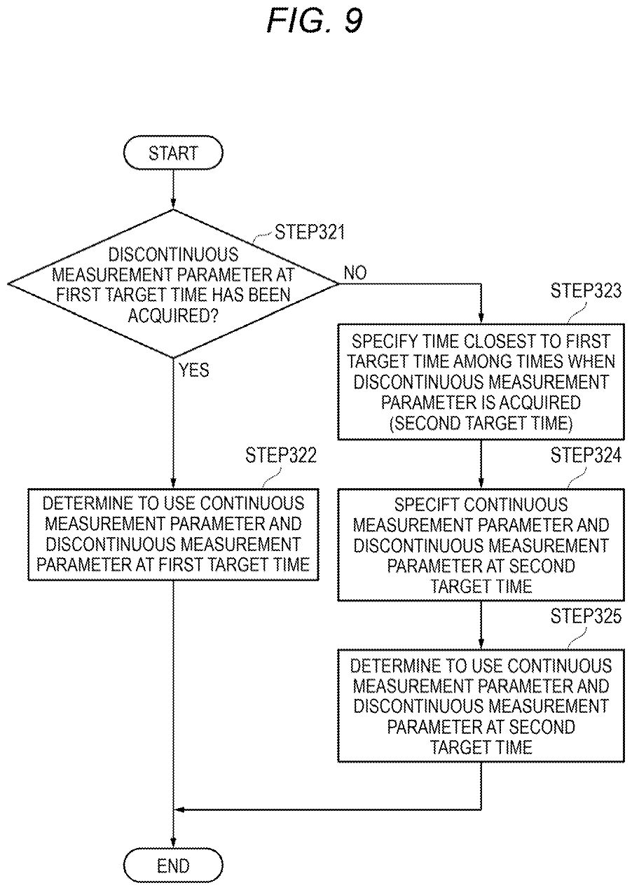

Next, a second example will be described. In description of the second example, description overlapping with the first example will be omitted. The second example is different from the first example only in a method of specifying the continuous measurement parameter and the discontinuous measurement parameter used in the first discrimination method performed in STEP 03 of FIG. 6. Therefore, the method of specifying the physiological information used in the first discrimination method in the second example will be described with reference to FIG. 9.

STEPS 321 to 323 of FIG. 9 are the same as STEPS 311 to 313 of FIG. 8.

In a situation where the discontinuous measurement parameter is not acquired at the time B which is the first target time (see FIG. 7B), the controller 4 specifies the continuous measurement parameter and the discontinuous measurement parameter at the time A when the time A is specified as the second target time (STEP 324). When the continuous measurement parameter and the discontinuous measurement parameter at the time A are specified, the controller 4 determines to use the continuous measurement parameter and the discontinuous measurement parameter at the time A in the first determination method (STEP 325). In this way, in the second example, when the discontinuous measurement parameter is not acquired at the first target time, not only the discontinuous measurement parameter but also the continuous measurement parameter acquired at the second target time is used in the first discrimination method.

According to the above configuration, even when the first target time, which is the reference time among the target times set arbitrarily, is the time when the discontinuous measurement parameter is not measured, the generator generates the condition information indicating the condition of the subject based on the continuous measurement parameter and the discontinuous measurement parameter at the second target time which is the time closest to the first target time. Therefore, based on the continuous measurement parameter and the discontinuous measurement parameter acquired at the same time, the generator 41 can generate accurate condition information at that time point.

### Third Example

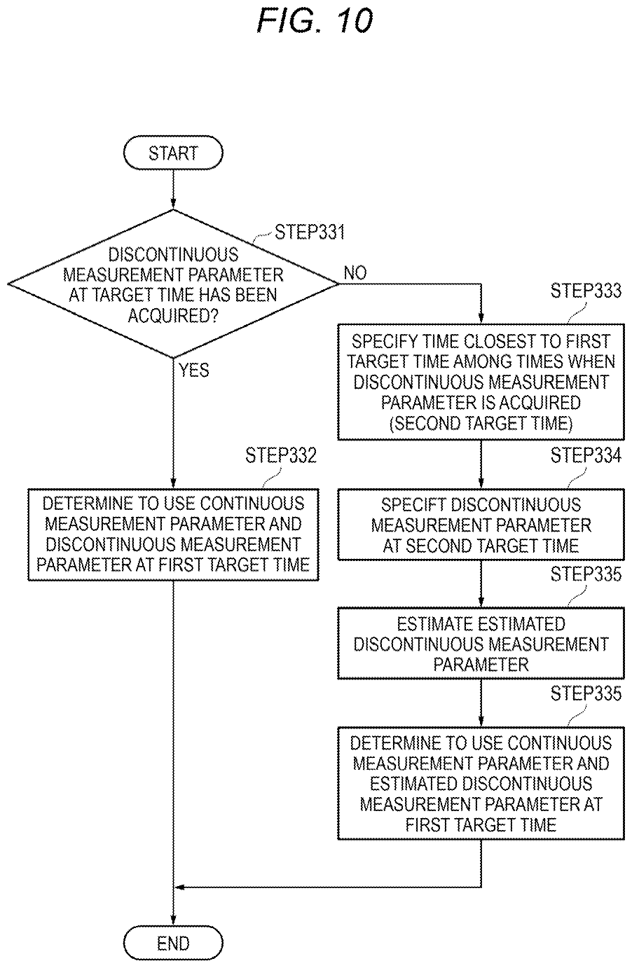

Next, a third example will be described. In description of the third example, description overlapping with the first example will be omitted. The third example is different from the first example only in the method of specifying the continuous measurement parameter and the discontinuous measurement parameter used in the first discrimination method performed in STEP 03 of FIG. 6. Therefore, the method of specifying the physiological information used in the first discrimination method in the third example will be described with reference to FIG. 10.

STEPS 331 to 333 of FIG. 10 are the same as STEPS 311 to 313 of FIG. 8.

In the situation where the discontinuous measurement parameter is not acquired at the time B which is the first target time (see FIG. 7B), the controller 4 specifies the discontinuous measurement parameter at the time A when the time A is specified as the second target time (STEP 334). When the discontinuous measurement parameter at the time A is specified, the estimator 42 estimates the estimated discontinuous measurement parameter at the first target time (time B), which is the time when the discontinuous measurement parameter is not acquired, based on the specified discontinuous measurement parameter (STEP 335). The estimator 42 can estimate the estimated discontinuous measurement parameter based on, for example, trend information based on the discontinuous measurement parameter acquired by the second acquisition unit 22 and the discontinuous measurement parameter acquired at the second target time.

When the estimated discontinuous measurement parameter is estimated, the controller 4 determines to use the continuous measurement parameter and the estimated discontinuous measurement parameter at the first target time in the first discrimination method (STEP 336). In this way, in the third example, when the discontinuous measurement parameter is not acquired at the first target time, the estimated discontinuous measurement parameter at the first target time is estimated based on the discontinuous measurement parameter acquired at the second target time, and the continuous measurement parameter and the estimated discontinuous measurement parameter at the first target time are used in the first discrimination method.

According to the above configuration, even when the discontinuous measurement parameter is not acquired at the first target time, the estimated discontinuous measurement parameter is estimated by the estimator 42 as an estimated value at the first target time. Therefore, even if the discontinuous measurement parameter is not acquired at the first target time, the generator 41 can generate the condition information.

### Second Embodiment

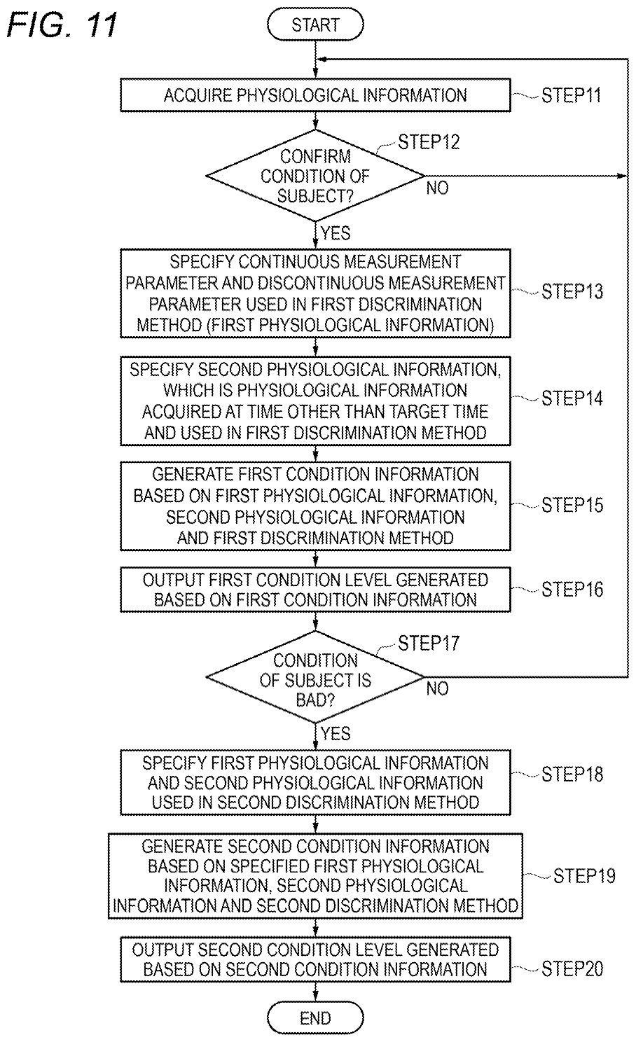

Next, a second embodiment will be described with reference to FIGS. 11, 12A and 12B. FIG. 11 is a flowchart illustrating the method of generating the condition information and the condition level information. FIG. 12A and FIG. 12B illustrate the acquisition status of the physiological information. The second embodiment is different from the first embodiment in that a plurality of pieces of physiological information are used in the first discrimination method and the second discrimination method. For example, even if a score indicating that the condition of the subject is good is calculated since physiological information indicating that the condition of the subject is good is acquired at the target time momentarily, the condition of the subject may still be bad in a certain period including the target time. On the contrary, for example, even if a score indicating that the condition of the subject is bad is calculated since physiological information indicating that the condition of the subject is bad is acquired at the target time momentarily, the condition of the subject may still be good in a certain period including the target time. Therefore, in the second embodiment, not only the physiological information acquired at the target time but also physiological information acquired at a time D (9:20 on Jul. 10, 2017) different from the target time is used to generate the condition information and the condition level information. In this way, in the second embodiment, the condition information and the condition level information are generated also in consideration of a trend of the condition of the subject during a certain period. It should be noted that description overlapping with the first embodiment is omitted.

STEPS 11 and 12 of FIG. 11 are the same as STEPS 01 and 02 of FIG. 6.

Here, for example, when the acquisition status of the physiological information is as illustrated in FIG. 12A, the discontinuous measurement parameter is acquired at the time B and the time D. Meanwhile, the continuous measurement parameter is continuously acquired not only at the time B and the time D but also at other times. When the acquisition status of the physiological information is as illustrated in FIG. 12B, the discontinuous measurement parameter is acquired at the time A and the time D. Meanwhile, the continuous measurement parameter is continuously acquired not only at the time A and the time D but also at other times. In the present embodiment, the physiological information including the continuous measurement parameter and the discontinuous measurement parameter acquired at the time B in a situation illustrated in FIG. 12A, or the physiological information including the continuous measurement parameter acquired at the time B and the discontinuous measurement parameter acquired at the time A in a situation illustrated in FIG. 12B is referred to as the first physiological information.

As illustrated in FIG. 11, when the ward nurse determines that it is necessary to confirm the condition of the subject (Kodentaro) (YES in STEP 12), the controller 4 specifies the continuous measurement parameter and the discontinuous measurement parameter used in the first discrimination method (STEP 13). The continuous measurement parameter and the discontinuous measurement parameter used in the first discrimination method are specified by any one of the methods described in the first example to the third example. The physiological information specified in STEP 13 is an example of the first physiological information.

After the first physiological information is specified, the controller 4 specifies physiological information (an example of the second physiological information) which is acquired at a time other than the target time (that is, the time B which is the first target time and the time A which is the second target time) and is different from the first physiological information used in the first discrimination method (STEP 14). As illustrated in FIGS. 12A and 12B, in the present embodiment, the continuous measurement parameter and the discontinuous measurement parameter are acquired at the time D which is different from the first target time and the second target time. Therefore, the second physiological information specified in STEP 14 includes the continuous measurement parameter and the discontinuous measurement parameter acquired at the time D.

After the second physiological information is specified, the generator 41 generates the first condition information (total score A) based on the first physiological information and second physiological information, and the first discrimination method (STEP 15). The total score A generated in STEP 15 and the total score B generated in STEP 19 to be described later below are, for example, an average score of a total score based on the first physiological information and a total score based on the second physiological information, or a higher or lower score among the total score based on the first physiological information and the total score based on the second physiological information.

STEPS 16 and 17 are the same as STEPS 05 and 06 of FIG. 6.

When it is determined that the condition of Kodentaro is bad (YES in STEP 17), the ward nurse requests the certified nurse or doctor to perform the further condition discrimination. The certified nurse or doctor performs the input operation described above. When the input operation is performed, the additional discrimination information is transmitted to the controller 4. Upon receiving the additional discrimination information, the controller 4 specifies the first physiological information and the second physiological information used in the second discrimination method (STEP 18). That is, upon receiving the additional discrimination information, the controller 4 specifies the physiological information at the target time specified in STEP 13 (an example of the first physiological information) and the physiological information at an acquisition time (time D) of the second physiological information specified in STEP 14 (an example of the second physiological information). The specified physiological information not only includes the physiological information used in the first discrimination method, but also includes other physiological information (for example, heart rate or body temperature) used in the second discrimination method.

For example, when the acquisition status of the physiological information is as illustrated in FIG. 12A, the controller 4 specifies the continuous measurement parameter and the discontinuous measurement parameter at the time B and the time D. When the acquisition status of the physiological information is as illustrated in FIG. 12B, the controller 4 specifies the continuous measurement parameter at the time B and the time D, and the discontinuous measurement parameter at the time A and the time D.

After the first physiological information and the second physiological information used in the second discrimination method are specified, the generator 41 generates the second condition information (total score B) based on the specified first physiological information and second physiological information, and the second discrimination method (STEP 19).

STEP 20 is the same as STEP 09 of FIG. 6.

According to the above configuration, the generator 41 generates the condition information based on the physiological information at the plurality of times including the target time such as the time A and the time B, and the time D which is different from the target time. Therefore, the condition information generation device 1 can also generate the condition information indicating the condition of the subject during the certain period (in the present embodiment, from 9:20 to 9:40 on Jul. 10, 2017). For example, in the situation shown in FIG. 12A, when the total score calculated based on the first physiological information acquired at the time B indicates that the condition of the subject is good while the total score calculated based on the second physiological information acquired at the time D indicates that the condition of the subject is bad, the condition information generation device 1 can generate the condition information indicating that the condition of the subject is bad. More specifically, for example, when the generator 41 generates the average score of the total score based on the first physiological information and the total score based on the second physiological information as the condition information, the total score A based on the first physiological information is 0, the total score A based on the second physiological information is 2, the total score B based on the first physiological information is 0, and the total score B based on the second physiological information is 6, the generator 41 generates the first condition information and the second condition information indicating that the condition of the subject is bad. In this way, for example, even in a case where a total score indicating that the condition of the subject is good is calculated as a result of acquisition of physiological information indicating that the condition of the subject is momentarily good at the target time despite the condition of the subject is bad during a certain period, the condition information generation device 1 can generate the condition information in consideration of the trend of the condition of the subject during the certain period.

The embodiments described above are intended to facilitate understanding of the presently disclosed subject matter, and are not intended to limit the presently disclosed subject matter. The presently disclosed subject matter can also be modified and improved without departing from the spirit thereof.

Although the method of specifying the continuous measurement parameter and the discontinuous measurement parameter used in the first discrimination method is exemplified in the first example to the third example in the presently disclosed subject matter, which of the first example to the third example is to be used may be appropriately set by the medical worker, or automatically set by the controller 4 included in the condition information generation device 1 based on a condition set in advance by the medical worker.

Although the generator 41 outputs the output signal OS corresponding to the condition level information to the output interface 5 in the presently disclosed subject matter, the output signal OS corresponding to the condition information or the output signal OS corresponding to the condition information and the condition level information may also be output to the output interface 5. When the output signal OS corresponding to the condition information is output to the output interface 5, a numerical value (total score A or total score B) based on the condition information is displayed on the display unit of the external device 10 instead of the color indicating the condition level of the subject on the display unit of the external device 10. When the output signal OS corresponding to the condition information and the condition level information is output to the output interface 5, the display unit of the external device 10 displays the numerical value based on the condition information and the color indicating the condition level of the subject.

Although the generator 41 generates the condition information through using the condition discrimination method in the presently disclosed subject matter, the condition information may also be generated without using the condition discrimination method.

Although the ward nurse determines whether the condition of the subject is bad based on the condition level information of the subject displayed on the display unit of the external device 10 and requests the certified nurse or doctor to perform further condition discrimination when the condition is bad in STEP 06 of FIG. 6 or STEP 17 of FIG. 11 in the presently disclosed subject matter, the presently disclosed subject matter is not limited thereto. For example, the controller 4 may determine whether the condition of the subject is bad based on the condition information or the condition level information, and the condition information generation device 1 may notify the certified nurse or doctor of the condition when the condition of the subject is bad. The method of notifying the certified nurse or doctor is, for example, a method of sending a message to a communication terminal carried by the certified nurse or doctor. The condition information generation device 1 may also include a display unit and display state level information on the display unit.

Although the first condition level information and the second condition level information are generated based on the standardization table P in the presently disclosed subject matter, the first condition level information and the second condition level information may also be generated based on the standardization table Q.

In the second embodiment, the discontinuous measurement parameter at the time B may not be the discontinuous measurement parameter acquired at the time B, and may also be, for example, the estimated discontinuous measurement parameter at the time B estimated based on the discontinuous measurement parameter at the time A.

Although the generator 41 generates the first condition information based on the first physiological information and the second physiological information in the second embodiment, the presently disclosed subject matter is not limited thereto. For example, the generator 41 may also generate the first condition information based on the first physiological information, the second physiological information, and other physiological information acquired at times other than the target time and an acquisition time of the second physiological information.

Although the generator 41 generates the second condition information based on the first physiological information and the second physiological information in the second embodiment, the presently disclosed subject matter is not limited thereto. For example, the generator 41 may also generate the second condition information based on the first physiological information, the second physiological information, and other physiological information acquired at times other than the target time and the acquisition time of the second physiological information.

The aforementioned embodiments are summarized as follows.

A condition information generation device according to a first aspect of the presently disclosed subject matter includes: an acquisition unit configured to acquire physiological information of a subject; and a generator configured to generate condition information of the subject based on the physiological information. The generator is configured to generate the condition information based on the physiological information acquired at a target time that is set arbitrarily, the target time being a time that is earlier than a time when the condition information generation device generates the condition information.

A condition information generation method according to a second aspect of the presently disclosed subject matter includes: acquiring physiological information of a subject, and generating condition information of the subject based on the physiological information. The generating comprises generating the condition information based on the physiological information acquired at a target time that is set arbitrarily, the target time being a time that is earlier than a time when the condition information is generated.

A non-transitory computer-readable medium according to a third aspect of the presently disclosed subject matter stores a computer program for causing a computer to execute the condition information generation method according to the second aspect of the presently disclosed subject matter.

According to the configurations described above, for example, when the condition of the subject is discriminated after the time when the medical worker determines that the condition of the subject may have deteriorated, the generator can generate the condition information indicating the condition of the subject at any target time set arbitrarily, such as the time when the medical worker determines that the condition of the subject may have deteriorated.

According to the presently disclosed subject matter, the condition information generation device, the condition information generation method, and the non-transitory computer-readable medium can be provided to enable the indication of the condition at the time earlier than the time when the condition information of the subject is generated, such as the time when the medical worker determines that the condition of the subject may have deteriorated.

What is claimed is:

1. A condition information generation device comprising:
an acquisition unit configured to acquire physiological information of a subject; and
a generator configured to generate condition information of the subject based on the physiological information, wherein
the generator is configured to generate the condition information based on the physiological information acquired at a target time that is set arbitrarily, the target time being a time that is earlier than a time when the condition information generation device generates the condition information.

2. The condition information generation device according to claim 1, wherein the physiological information includes: a continuous measurement parameter of the subject that is temporally continuously measurable; and a discontinuous measurement parameter of the subject that is measurable at arbitrary timings,
the acquisition unit includes: a first acquisition unit configured to acquire the continuous measurement parameter; and a second acquisition unit configured to acquire the discontinuous measurement parameter, and
the generator is configured to generate the condition information based on the continuous measurement parameter and the discontinuous measurement parameter.

3. The condition information generation device according to claim 2, wherein the target time includes: a first target time, which is a reference time; and a second target time, which is a time closest to the first target time among times when the discontinuous measurement parameter is acquired, and
the generator is configured to generate the condition information based on the continuous measurement parameter acquired by the first acquisition unit at the first target time and the discontinuous measurement parameter acquired by the second acquisition unit at the second target time.

4. The condition information generation device according to claim 2, wherein the target time includes: a first target time, which is a reference time; and a second target time, which is a time closest to the first target time among times when the discontinuous measurement parameter is acquired, and
the generator is configured to generate the condition information based on the discontinuous measurement parameter acquired by the second acquisition unit at the second target time and the continuous measurement parameter acquired by the first acquisition unit at the second target time.

5. The condition information generation device according to claim 2, wherein the target time includes: a first target time, which is a reference time; and a second target time, which is a time closest to the first target time among times when the discontinuous measurement parameter is acquired,
the condition information generation device further comprising: an estimator configured to estimate an estimated discontinuous measurement parameter at the first target time based on the discontinuous measurement parameter acquired by the second acquisition unit at the second target time, and
the generator is configured to generate the condition information based on the continuous measurement parameter at the first target time and the estimated discontinuous measurement parameter at the first target time.

6. The condition information generation device according to claim 1, wherein the generator is configured to generate the condition information based on a discrimination method for discriminating a condition of the subject.

7. The condition information generation device according to claim 6, wherein the discrimination method includes: a first discrimination method; and a second discrimination method different from the first discrimination method,
the condition information includes: first condition information generated based on the first discrimination method; and second condition information generated based on the second discrimination method, and
the second condition information is generated based on physiological information acquired at a same time as a time when physiological information used to generate the first condition information is acquired, the second condition information being generated at a time temporally later than a time when the first condition information is generated.

8. The condition information generation device according to claim 7, wherein the first discrimination method is a discrimination method based on qSOFA (quick Sequential Organ Failure Assessment), and the second discrimination method is a discrimination method based on NEWS (National Early Warning Score).

9. The condition information generation device according to claim 1, wherein the generator is configured to generate the condition information based on first physiological information acquired at the target time and second physiological information acquired at a time different from the target time.

10. A condition information generation method, comprising:
acquiring physiological information of a subject, and
generating condition information of the subject based on the physiological information, wherein
the generating comprises generating the condition information based on the physiological information acquired at a target time that is set arbitrarily, the target time being a time that is earlier than a time when the condition information is generated.

11. A non-transitory computer-readable medium that stores a computer program for causing a computer to execute the condition information generation method according to claim 10.
